(19) United States
(12) Patent Application Publication
(10) Pub. No.: US 2011/0091356 A1
(43) Pub. Date: Apr. 21, 2011

(54) MICRO-FLUIDIC DEVICE AND SAMPLE TESTING METHOD USING THE SAME

(75) Inventors: Kui Hyun KIM, Hwaseong-si (KR); Beom Seok LEE, Hwaseong-si (KR); In Wook KIM, Seongnam-si (KR); Ji Won KIM, Suwon-si (KR); Yang Ui LEE, Seoul (KR)

(73) Assignee: SAMSUNG ELECTRONICS CO., LTD., Suwon-si (KR)

(21) Appl. No.: 12/884,762

(22) Filed: Sep. 17, 2010

(30) Foreign Application Priority Data
Oct. 20, 2009 (KR) 10-2009-099957

(51) Int. Cl. G01N 33/00

(52) U.S. Cl. 422/68.1

(57) ABSTRACT

A micro-fluidic device containing an anti-coagulant and a sample testing apparatus equipped with the same are provided. The micro-fluidic device includes a sample chamber which receives a sample, an anti-coagulant chamber which receives an anti-coagulant, a channel which communicably connects the sample chamber to the anti-coagulant chamber, and a valve which opens and closes the channel.

### CROSS-REFERENCE TO RELATED APPLICATION

This application claims priority from Korean Patent Application No. 10-2009-099957, filed on Oct. 20, 2009 with the Korean Intellectual Property Office, the entire disclosure of which is incorporated herein by reference.

### BACKGROUND

1. Field

Apparatuses and methods consistent with embodiments relate generally to a micro-fluidic device and a sample testing apparatus using the same and, more particularly, to a micro-fluidic device containing an anti-coagulant and a sample testing apparatus equipped with the same.

2. Description of the Related Art

In general, a micro-fluidic device conducts biological or chemical reactions by operating upon a small amount of fluid. Such a micro-fluidic device has a micro-fluidic structure provided in a platform in different forms or shapes such as a chip, a disk, etc.

The micro-fluidic structure generally has a chamber to receive a fluid therein, a channel through which the fluid passes or flows and a valve to control the fluid flow. The chamber, channel and valve are combined and arranged according to different assembly designs.

In order to conduct various experiments including biochemical reactions on a chip, a micro-fluidic structure is arranged on a chip-type platform in what is referred to as a “biochip.” Especially, a device fabricated for multi-stage treatment and/or operation on a single chip is referred to as a “lab-on-a-chip.”

In order to flow and shift a fluid in a micro-fluidic structure of a micro-fluidic device, a driving pressure is generally required. The driving pressure may be a capillary pressure or pressure generated using an alternative pump. Also, a micro-fluidic structure may be arranged on a disk-type platform to generate a centrifugal force to cause the movement of a fluid.

With the micro-fluidic device described above, a sample of whole blood, serum, plasma, etc may be analyzed. In this regard, such sample materials should be anti-coagulative, otherwise, blood is coagulated over time. Therefore, before introducing a sample obtained from whole blood, plasma, etc. into a micro-fluidic device, the sample must be subjected to anti-coagulant treatment as a pre-treatment.

When using the sample obtained from whole blood, plasma, etc., an alternative tool, such as a tube, is used for anti-coagulant treatment, the tube being an undesirable expense. Furthermore, pre-treatment is an additional step in a process for analyzing a sample, thus extending the time required to inspect a sample.

In order to solve the foregoing problems, technologies for omitting an anti-coagulation process as pre-treatment of whole blood or the like by, for example, introducing a lyophilized anti-coagulant into a micro-fluidic device, coating a surface of the micro-fluidic device with an anti-coagulant, and so forth, have been developed.

However, although some inspection items require different anti-coagulants, some techniques generally use a micro-fluidic device containing a single particular anti-coagulant. That is, it is a drawback to use a single micro-fluidic device with only one anti-coagulant, as a variety of inspection items cannot be tested simultaneously.

Moreover, the foregoing techniques entail a problem in that, since a sample injected into the device generally reacts with the anti-coagulant, the other sample(s) not requiring anti-coagulant treatment cannot undergo inspection.

### SUMMARY

Exemplary embodiments provide a micro-fluidic device containing an anti-coagulant and a sample testing apparatus equipped with the same.

One or more exemplary embodiments provide a micro-fluidic device containing an anti-coagulant, which can perform an anti-coagulant treatment in a single device, as well as a sample testing apparatus using the foregoing micro-fluidic device.

One or more exemplary embodiments also provide a micro-fluidic device containing an anti-coagulant and a sample testing apparatus using the same which can inspect different items from a particular sample in a single device.

One or more exemplary embodiments provide a micro-fluidic device containing an anti-coagulant and a sample testing apparatus using the same, which can inspect a plurality of samples in a single device.

According to an aspect of an exemplary embodiment, there is provide a micro-fluidic device including: a sample chamber which receives a sample; an anti-coagulant chamber which receives an anti-coagulant; a channel which communicably connects the sample chamber to the anti-coagulant chamber; and a valve which opens and closes the channel.

The micro-fluidic device may be a disk-shaped platform.

When the valve is open, the micro-fluidic device rotates to generate a centrifugal force and shift the anti-coagulant from the anti-coagulant chamber to the sample chamber.

When the valve is open, the anti-coagulant in the anti-coagulant chamber may be admixed with the sample in the sample chamber.

The valve may include a phase transition material which is in a solid state at room temperature and is transformed into a liquid phase when heated to open the valve upon the application of heat.

The valve may further include a micro-exothermic material which is dispersed in the phase transition material and absorbs electromagnetic radiation to emit heat energy.

The sample chamber has an inlet to receive the sample.

A plurality of anti-coagulant chambers may be provided, each anti-coagulant chamber in fluid communication with a respective sample channel, where the number of channels corresponds to the number of multiple anti-coagulant chambers.

At least two of the plurality of anti-coagulant chambers may contain different anti-coagulants.

The valve may be provided in each of the plurality of channels.

A plurality of sample chambers may be provided and, each of the anti-coagulant chambers may be in fluid communication with any one of the plurality of sample chambers.

In another exemplary embodiment, a plurality of sample chambers and a plurality of anti-coagulant chambers may be installed, wherein the plurality of channels communicably connect the plurality of sample chambers to the plurality of anti-coagulant chambers.

A plurality of valves may be provided in the plurality of channels.

At least two of the plurality of valves may be independently driven or operated.

At least two of the plurality of anti-coagulant chambers may contain different anti-coagulants.

The micro-fluidic device may further include a data region which stores information regarding types of anti-coagulant contained in the anti-coagulant chamber is stored.

The data region may contain barcode type data.

The valve may be normally closed.

According to an aspect of another exemplary embodiment, there is provided a micro-fluidic device including: a sample chamber which receives a sample; an anti-coagulant chamber which is communicably connected to the sample chamber and receives an anti-coagulant; and a valve provided between the sample chamber and the anti-coagulant chamber, wherein the valve is selectively opened to admix the anti-coagulant with the sample.

According to an aspect of another exemplary embodiment, there is provided a sample testing apparatus including: a micro-fluidic device which includes a sample chamber which receives a sample, an anti-coagulant chamber which is communicably connected to the sample chamber and receives an anti-coagulant, and a valve provided between the sample chamber and the anti-coagulant chamber, wherein the sample test apparatus further includes a valve-opening device which opens the valve, and a control unit to drive the valve-opening device when the micro-fluidic device is mounted on the apparatus, so as to selectively admix the sample in the sample chamber with the anti-coagulant from the anti-coagulant chamber.

The valve may further comprise a phase transition material which is transformed into a liquid phase when heated, while the valve opening device may be a heater to heat the phase transfer material.

The valve may contain a phase transition material and a micro-exothermic material which is dispersed in the phase transition material and absorbs electromagnetic radiation to emit heat energy, while the valve opening device may be a device generating electromagnetic radiation.

The micro-fluidic device may be a centrifugal disk-type micro-fluidic device and may further include a spindle motor to rotate the micro-fluidic device.

The control unit drives the spindle motor by centrifugal force when the valve is opened, so that the anti-coagulant of the anti-coagulant chamber flows into the sample chamber.

The sample testing apparatus may further include an input unit which inputs information on types of the samples injected into the sample chamber, while the control unit determines whether or not the anti-coagulant is required based on types of samples, and wherein the control unit operates the valve opening device based on the determined results.

A plurality of anti-coagulant chambers may be provided, wherein at least two of the plurality of anti-coagulant chambers receive different anti-coagulants. The sample testing apparatus has an input unit which inputs items to be tested with the micro-fluidic device. The control unit drives the valve opening device to admix one of the anti-coagulants with a corresponding sample according to inspection items input by the input unit.

The micro-fluidic device may further comprise a data region which stores information on a type of anti-coagulant contained in the anti-coagulant chamber, and a data reading unit to which extracts information stored in the data region as to the types of anti-coagulant.

As described above, the micro-fluidic device according to one aspect of an exemplary embodiment has an anti-coagulant chamber containing a liquid anti-coagulant, so as to eliminate alternative pre-treatment when inspection is implemented using a sample.

Further, the micro-fluidic device according to one aspect of an exemplary embodiment contains a plurality of different anti-coagulants in a single platform, enabling inspection of various samples and/or testing various inspection items of a sample.

### BRIEF DESCRIPTION OF THE DRAWINGS

The above and/or other aspects will become apparent and more readily appreciated from the following description of exemplary embodiments, taken in conjunction with the accompanying drawings, of which:

FIG. 1 is a perspective view illustrating a micro-fluidic device according to an exemplary embodiment;

FIGS. 2 and 3 are cross-sectional views illustrating an example of a closed valve;

FIG. 4 is a detailed view illustrating configurations of a micro-fluidic device according to another exemplary embodiment;

FIG. 5 is a detailed view illustrating a configuration of a micro-fluidic device according to another exemplary embodiment; and

FIG. 6 is a block diagram illustrating a sample testing apparatus using the micro-fluidic device according to any one of the exemplary embodiments.

### DETAILED DESCRIPTION

Hereinafter, a micro-fluidic device and a sample testing apparatus using the same according to exemplary embodiments will be described with reference to the accompanying drawings.

The same numerical symbols in the drawings refer to substantially the same configured elements. Separate structures such as a chamber, a channel, and the like are simply illustrated and dimensional ratios of the same may be different from real scales thereof, instead being enlarged or reduced. In expressions ‘micro-fluidic device,’ ‘micro-particle,’ etc., ‘micro’ are not limitedly construed as a size unit but used in contrast with ‘macro.’

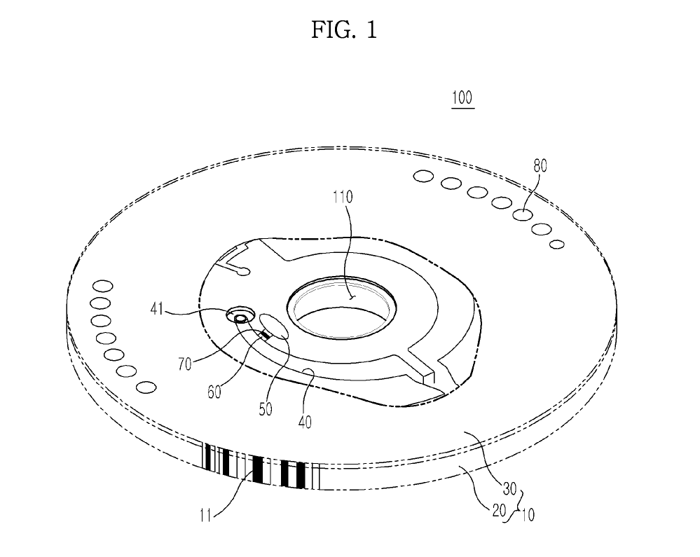

FIG. 1 is a perspective view illustrating a micro-fluidic device according to an exemplary embodiment.

This exemplary embodiment particularly describes a disk-type micro-fluidic device using centrifugal force to drive fluid movement, although a variety of micro-fluidic devices using capillary pressure or pump pressure as a driving pressure for fluid movement may also be employed.

Referring to FIG. 1, a micro-fluidic device 100 according to an exemplary embodiment has a rotational disk-type platform 10.

The platform 10 may be formed using plastic materials such as acryl, polydimethylsiloxane (PDMS), etc., each of which is easily formable and has a biologically inactive surface. However, a raw material for fabrication of the platform is not particularly limited and may include any materials with chemical or biological stability, optical transparency and/or mechanical workability.

The platform 10 may be formed of a multi-layered plate, and a chamber and a channel may be provided inside the platform by forming engraved structures corresponding to the chamber and the channel on a face at which one layer comes into contact with another layer, and then adhering these structures to the face.

The platform 10 may have, for example, a structure including a first plate 20 and a second plate 30 attached with the first plate, or a structure including a partition panel to define a channel through which a fluid flows as well as a chamber to receive the fluid between the first plate 20 and the second plate 30.

The first plate 20 may be attached to the second plate 30 using adhesive or a double-sided adhesive tape, or other methods including ultrasonic welding, laser welding, and the like.

The micro-fluidic device 100 includes at least one chamber to receive a fluid, at least one channel connected to the chamber to function as a fluid path, and a valve for opening and closing the channel so as to control a flow of the fluid. Such chamber, channel and/or valve are appropriately arranged for particular uses of the micro-fluidic device in biochemical applications, for example, centrifugation of a fluid specimen, immunoserum response, genetic analysis, gene extraction, gene amplification, and so forth. For instance, the micro-fluidic device according to one embodiment may have a chamber, a channel and a valve which are aligned with a number of designs in consideration of use thereof and a detailed description of particular arrangements thereof will be omitted for brevity.

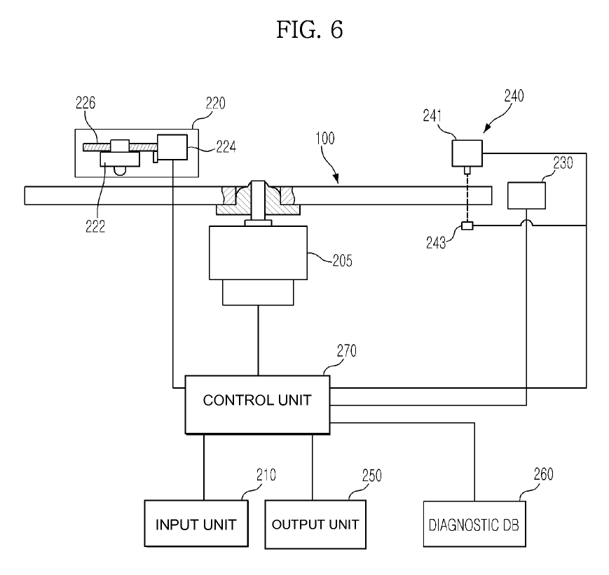

The micro-fluidic device 100 is provided in a disk form and is mounted on a spindle motor 205 (see FIG. 6) for high speed rotation. A fixation hole 110 is formed in the center of the micro-fluidic device 100 in order to fix the micro-fluidic device to the spindle motor 205. A fluid remaining in the chamber or channel of the micro-fluidic device 100 is forced toward an outer circumference (or a periphery) of the platform 10 by the centrifugal force generated in the platform 10 by rotation of the spindle motor 205.

In general, an “inner side” refers to a face near to a center of rotation of the platform 10 (i.e., near the fixation hole 110), while an “outer side” refers to a face far from the center of rotation (i.e., nearer to the outer circumference of the platform 10).

The micro-fluidic device 100 may include a sample chamber 40 to receive a sample, an anti-coagulant chamber 50 to receive a liquid anti-coagulant A, a channel through which the sample chamber 40 communicates with the anti-coagulant chamber 50, and a valve 70 to open and close the channel 60. In one exemplary embodiment, the valve is generally closed.

The sample chamber 40 is located inside the platform 10 and may have an inlet 41 through which the sample is injected into the sample chamber.

The sample chamber 40 may contain various types of samples including, for example, whole blood, serum, plasma, urine, saliva, etc.

Some samples contained in the sample chamber 40 should be firstly mixed with an anti-coagulant “A” in order to inhibit blood coagulation before inspection thereof.

TABLE 1

| Sample | Type of anti-coagulant | Inspection items |
|---|---|---|
| Whole blood | EDPA | CBC, ESR, HbA1C, etc. |
| | Heparin | Blood gas assay, cellular immunity |
| | | test, etc. |
| Serum | — | Clinical chemistry and |
| | | immunoserum assay |
| Plasma | Sodium citrate | Blood coagulation test |

In TABLE 1, some examples of sample types, inspection items to be tested using a corresponding sample and types of anti-coagulant required for inspection are listed.

As shown in TABLE 1, the anti-coagulant used for inspection varies in accordance with sample type. For instance, whole blood requires different kinds of anti-coagulants along with different inspection items, while serum does not demand any anti-coagulant during inspection.

According to an exemplary embodiment, in order to eliminate inconvenient anti-coagulation treatments before injecting the sample into the micro-fluidic device, the anti-coagulant is firstly introduced into the micro-fluidic device, and then, is admixed with the sample only if necessary.

The anti-coagulant chamber 50 which receives the anti-coagulant “A” is located at an innermost position on the platform 10. The anti-coagulant “A” may be injected into the anti-coagulant chamber 50, after the first and second plates 20 and 30 are attached to each other. Otherwise, after injecting the anti-coagulant A into the anti-coagulant chamber 50 on the first plate 20, the second plate 30 is attached to the first plate 30 in a method described above, in turn completing the anti-coagulant chamber 50.

The anti-coagulant chamber 50 is positioned on an inner face of the platform, nearer to the center of the platform as compared to the sample chamber 40, in order for the anti-coagulant A to flow from the anti-coagulant chamber 50 to the sample chamber 40 due to a centrifugal force generated by rotation of the micro-fluidic device 100.

Where a fluid in the micro-fluidic device 100 does not flow by centrifugal force, that is, is driven or operated by a capillary valve or other valves passively opened at a constant pressure, a position of the anti-coagulant chamber is not particularly restricted as described in the foregoing exemplary embodiment, but instead may be varied to preferably admix the anti-coagulant in the anti-coagulant chamber with the sample in the sample chamber.

The channel 60 connects the anti-coagulant chamber 50 to the sample chamber 40.

The valve 70 located on the channel 60 optionally opens the channel 60.

The valve 70 may be a micro-fluidic valve, for example, a valve passively opened by applying a constant pressure, such as a capillary valve, or a valve actively opened using external power or energy generated by action signals. The valve 70 used in this exemplary embodiment is a so-called “normally-closed valve” which closes the channel in order to prevent flow of a fluid until the valve absorbs electromagnetic radiation and is transformed.

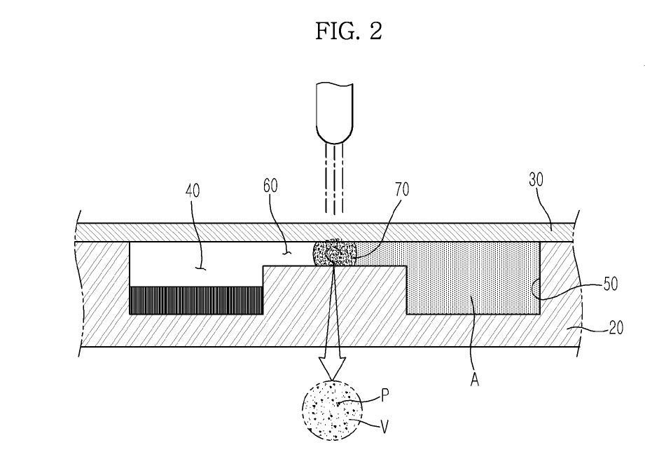

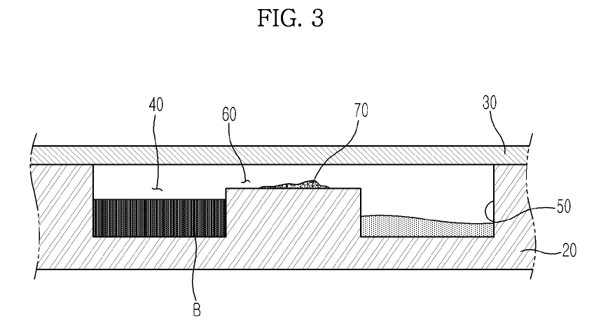

FIGS. 2 and 3 are cross-sectional views showing an example of a closed valve. The closed valve 70 may contain a valve material V. When the valve material V contained in the channel 60 is in a solid state is, the channel 60 is closed, as shown in FIG. 2. When the valve material V is fused at a high temperature, the valve material V flows into an inner space of the channel 60 thereby opening the channel 60. Then, as shown in FIG. 3, the fused material is again solidified as the channel 60 is in an open state.

The energy radiated by an external source may include electromagnetic radiation, and such external energy source may be selected from a laser source radiating a laser beam, a light emitting diode which radiates visible or infrared light, a xenon lamp, etc. In particular, the laser source may have at least one laser diode. The external energy source may be selected on the basis of electromagnetic radiation wavelengths absorbed by exothermic particles contained in the valve material V. Such valve material V may include thermoplastic resins such as cyclic olefin copolymer (COC), polymethylmethacrylate (PMMA), polycarbonate (PC), polystyrene (PS), polyoxymethylene (POM), perfluoralkoxy (PFA), polyvinylchloride (PVC), polypropylene (PP), polyethylene tetraphthalate (PET), polyetheretherketone (PEEK), polyamide (PA), polysulfone (PSU), polyvinylidene fluoride (PVDF), and the like. Alternatively, the valve material V may include a phase transition material in a solid state at room temperature. Such phase transition material may be wax. The wax is fused by heating and transformed into a liquid phase, in turn being expanded in volume. Such wax may include, for example, paraffin wax, microcrystalline wax, synthetic wax, natural wax, etc. The phase transition material may be a gel or thermoplastic resin. Such gel may include, for example, polyacrylamide, polyacrylate, polymethacrylate, polyvinylamide, etc. The valve material V may contain a micro-exothermic material P dispersed therein to absorb electromagnetic radiation and generate heat. Such exothermic material P may include numerous particles, each having a diameter of approximately 1 nanometer (nm) to 100 micrometers (μm), sufficient to freely pass through the micro-channel 60 having a length of 0.1 millimeters (mm) and a width of 1 mm. The micro-exothermic material “P” is heated to rapidly elevate temperature and generates heat when electromagnetic energy is provided by a laser, and is uniformly dispersed into the wax. In order to exhibit these features, the micro-exothermic material may have a core containing metal components and a hydrophobic surface structure. For instance, the micro-exothermic material may have an iron (Fe) based core and a specific molecular structure including plural surfactant components to be bonded to Fe in order to enclose the Fe. The micro-exothermic material may be stored in a dispersed state in a carrier oil. In order to uniformly disperse the micro-exothermic material having a hydrophobic surface structure in the carrier oil, such carrier oil may also be hydrophobic. After the carrier oil containing the micro-exothermic material dispersed therein is poured into a fused phase transition material and homogeneously admixed, this mixture is injected into the channel 60 and solidified, in turn blocking the channel 60. The micro-exothermic material is not particularly restricted to polymer particles described above, and may be in quantum dot or magnetic bead form. Such micro-exothermic material may include micro-metallic oxides such as aluminum oxide (Al₂O₃), titanium dioxide (TiO₂), tantalum oxide (Ta₂O₃), iron (III) oxide (Fe₂O₃), ferrous-ferric oxide (Fe₃O₄), hafnium oxide (HfO₂), and the like.

The closed valve 70 may not contain such micro-exothermic material, but contain the phase transition material alone without the micro-exothermic material. In this case, the sample testing apparatus may further include a heater to heat a corresponding valve to be opened, wherein the heater is located apart from the micro-fluidic device in a non-contact manner to heat and fuse the valve material, in turn opening the valve.

The valve 70 may be selectively opened in association with a sample contained in the sample chamber 40. When the valve 70 is open, the micro-fluidic device 100 rotates and the anti-coagulant A in the anti-coagulant chamber 50 flows into the sample chamber 40 and is admixed with the sample in the sample chamber 40 to form a mixed solution “B.”

A dilution chamber (not shown) to receive a diluent may be placed outside of the sample chamber 40 and at least one reaction chamber 80 may be provided outside of the dilution chamber. Each reaction chamber may receive a liquid or dried solid reagent.

A data region 11 may be positioned on a periphery of the micro-fluidic device 100.

The data region 11 may include information on types of anti-coagulant A contained in the anti-coagulant chamber 50, and such information may be stored in a barcode form.

Although the data region 11 described in the foregoing exemplary embodiment is positioned on the periphery of the micro-fluidic device 100, the data region 11 may be placed on a top or bottom of the micro-fluidic device 100, or inside thereof.

The barcode form described above may be a primary barcode or selected from other barcode types and matrix codes (i.e., secondary barcode) (not shown) in order to store large quantities of information.

A variety of necessary information including, for example, the type of anti-coagulant contained in the anti-coagulant chamber 50, information for validity of the micro-fluidic device, identifiable information such as serial number, and so forth may be stored in the data region 11.

Although the foregoing exemplary embodiment describes the data region consisting of barcodes, other substances for storage of information may be used to form the data region 11, such as holograms, radio frequency identification (RFID) tags, and memory chips. In this embodiment, the sample testing apparatus may have a data reading unit 230 (FIG. 6) equipped with a reader to read the information stored in the data region having any specific configuration as described above.

When the data region is fabricated using a storage medium for reading/writing information such as memory chips, the data region may include additional information, including sample assay results, patient information, dates and times for blood sampling and inspection, whether or not to conduct inspection, etc. as well as identifiable information described in the exemplary embodiment.

Accordingly, when the micro-fluidic device is loaded on the sample testing apparatus, the sample testing apparatus detects the data region 11 of the micro-fluidic device 100 and identifies the type of anti-coagulant received in the micro-fluidic device 100. The sample testing apparatus opens the valve 70 if an anti-coagulant is required, which depends on the injected sample. Thus, the anti-coagulant may be admixed with the sample.

Next, a detailed description will be given of a micro-fluidic device according to another exemplary embodiment.

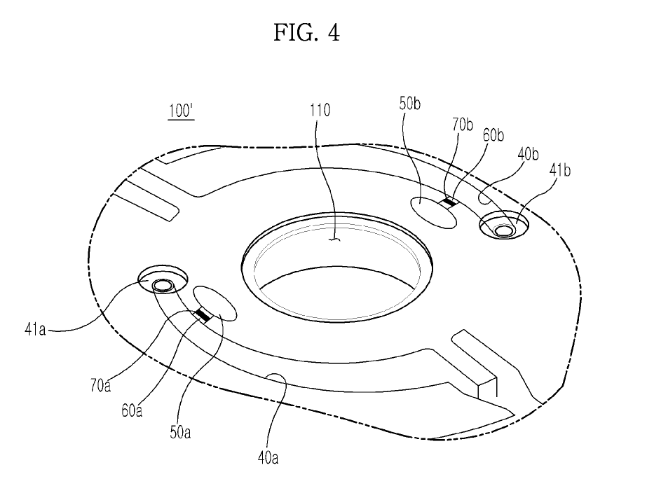

FIG. 4 is a detailed perspective view showing configurations of a micro-fluidic device according to another exemplary embodiment.

Compared to the previously described exemplary embodiment (referred to as ‘first embodiment’), the present exemplary embodiment describes a plurality of sample chambers, anti-coagulant chambers and channels for connection of these chambers, thus being distinguishable from the first embodiment. Hereinafter, with regard to the same configurations as described in the first embodiment, the same numerical symbols are used and a detailed description thereof will be omitted for brevity.

According to the present exemplary embodiment (referred to as ‘second embodiment’), a micro-fluidic device 100′ includes a pair of first and second sample chambers 40a and 40b spaced at an interval, while first and second anti-coagulant chambers 50a and 50b corresponding to the above sample chambers 40a and 40b, respectively, are provided inside of the same.

The sample chambers 40a and 40b have inlets 41a and 41b, which communicate with the anti-coagulant chambers 50a and 50b, respectively, through channels 60a and 60b. These channels 60a and 60b have valves 70a and 70b, respectively.

Compared to the micro-fluidic device configured with a sample chamber, an anti-coagulant chamber, a channel and a valve according to the exemplary embodiment of FIG. 1, the micro-fluidic device according to the exemplary embodiment of FIG. 4 includes pairs of such separate components arranged on opposite orientations of the device. However, two or more of each of these components, that is, the sample chamber, the anti-coagulant chamber, the channel and the valve, may also be provided. Here, two or more anti-coagulant chambers may receive different types of anti-coagulants.

According to the configuration described above, even multiple samples requiring different anti-coagulants may be subjected to one inspection process implemented in a single micro-fluidic device 100′ by introducing the multiple samples into the sample chambers 40a and 40b and opening the first and second valves 70a and 70b.

Where a sample requiring an anti-coagulant is introduced into the first sample chamber 40a and another sample requiring no anti-coagulant is introduced into the second sample chamber 40b, one of the paired valves 70a is opened to selectively admix the sample requiring the anti-coagulant with the anti-coagulant while the other of the valves 70b remains closed.

Alternatively, if both samples injected into the first and second sample chambers 40a and 40b do not require an anti-coagulant, sample inspection may be conducted while the pair of valves 70a and 70b is completely closed.

In this regard, a control unit 270 (FIG. 6) may determine whether the anti-coagulant is required or not based on information on sample type stored in an input unit 210 (FIG. 6) described below.

Next, a detailed description will be given of a micro-fluidic device according to a further exemplary embodiment.

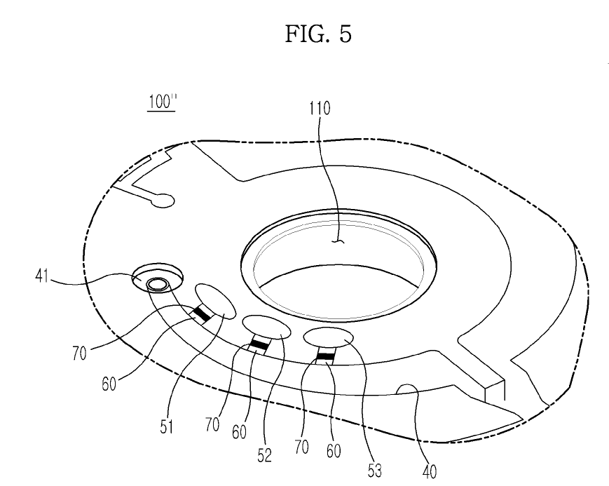

FIG. 5 is a detailed perspective view showing a configuration of a micro-fluidic device 100″ according to a further exemplary embodiment.

Compared to the exemplary embodiment of FIG. 1, the present exemplary embodiment describes a single sample chamber as well as a plurality of anti-coagulant chambers connected to the sample chamber, which are different from the first embodiment, while the other configurations are substantially identical to the exemplary embodiment of FIG. 1. Hereinafter, with regard to the same configurations as described in the exemplary embodiment of FIG. 1, the same numerical symbols are endowed and a detailed description thereof will be omitted for brevity.

According to the present exemplary embodiment, a micro-fluidic device 100″ includes a sample chamber 40 and a plurality of anti-coagulant chambers 51, 52 and 53 placed inside of the sample chamber 40. Although three anti-coagulant chambers 51, 52 and 53 are shown, two or more anti-coagulant chambers may be placed in the micro-fluidic device.

Such anti-coagulant chambers 51, 52 and 53 may receive two or more different anti-coagulants, one in each chamber. For example, a first anti-coagulant chamber 51 includes ethylenediaminetetraacetate (EDTA), a second anti-coagulant chamber 52 includes heparin and a third anti-coagulant chamber 53 receives sodium citrate.

According to such configurations, when whole blood is introduced into the sample chamber 40 and a blood gas assay is selected among inspection items, a second valve 72 is opened to admix heparin as an anti-coagulant required for the blood gas assay with the sample. In another example, if plasma is injected into the sample chamber 40 and a blood coagulation test is selected among inspection items, a third valve 73 is opened to admix sodium citrate as an anti-coagulant required for the blood coagulation test with the sample. Likewise, where serum is fed into the sample chamber 40, all of the valves 71, 72 and 73 remain closed since the serum does not need any anti-coagulant.

That is, by connecting the sample chamber 40 of the micro-fluidic device 100″ to multiple anti-coagulant chambers 51, 52 and 53 which receive different anti-coagulants, respectively, a single micro-fluidic device may be satisfactory to conduct various inspections.

A detailed description of a sample testing apparatus for sample inspection, using any one of the micro-fluidic devices according to the foregoing exemplary embodiments is provided below.

FIG. 6 is a block diagram of a sample testing apparatus using the micro-fluidic device according to an exemplary embodiment.

A sample testing apparatus according to an exemplary embodiment includes a spindle motor 205 to rotate a micro-fluidic device 100, a data reading unit 230, a valve opening device 220, an inspection unit 240, an input unit 210, an output unit 250, a diagnostic database (DB) 260, and a control unit 270 controlling individual components described above.

The spindle motor 205 rotates the micro-fluidic device 100, and also stops and rotates the micro-fluidic device 100 in order to move the micro-fluidic device 100 to a desired position.

Although not illustrated, the spindle motor 205 may have a motor-driving device to adjust an angular position of the micro-fluidic device. For example, the motor driving device may be a stepper motor or a DC motor.

The data reading unit 230 may be, for example, a barcode reader. According to the foregoing exemplary embodiment, the data reading unit 230 is arranged in parallel to the micro-fluidic device 100 and positioned apart from a periphery of the micro-fluidic device 100 at a constant interval so as to emit light to a data region 11 (i.e., barcode), which is provided on the periphery of the micro-fluidic device, and to receive light reflected from the data region 11. However, the data reading unit 230 may also be provided on a top or bottom of the micro-fluidic device 100.

The data reading unit 230 reads data stored in the data region 11 and transfers the read data to the control unit 270. The control unit 270 operates separate components of the micro-fluidic device based on the read data, in turn driving the sample testing apparatus.

The valve opening device 220 opens or closes multiple valves 70 of the micro-fluidic device 100, and includes an external energy source 222 as well as movement units 224 and 226 to shift the external energy source to any valve required to be opened.

The external energy source 222 may be a laser source radiating a laser beam, a light emitting diode radiating visible or infrared light, a xenon lamp, etc. In particular, the laser source may have at least one laser diode (LD).

Each of the movement units 224 and 226 is provided to adjust a position of the external energy source 222 so as to concentrate energy radiation toward a desired area of the micro-fluidic device, that is, the valve. Such movement unit may include a driving motor 224 and a gear unit 226 equipped with the external energy source 222 to move the external energy source 222 to a position above a valve to be opened by rotation of the driving motor 224.

The inspection unit 240 may include at least one light emission unit 241 and at least one light receiving unit 243 which corresponds to the light emission unit 241 and receives light penetrating a reaction chamber 80 of the micro-fluidic device 100.

The light emission unit 241 may be a flashing light source with a specific frequency including, for example, a semiconductor light emitting device such as a light emitting diode (LED) or an LD, a gas discharge lamp such as a halogen lamp or a xenon lamp, etc.

The light emission unit 241 is placed on a site at which light emitted from the light emission unit 241 passes through the reaction chamber 80 and reaches the light receiving unit 243.

The light receiving unit 243 generates electrical signals according to an intensity of incident light and adopts, for example, a depletion layer photodiode, an avalanche photodiode (APD), a photomultiplier tube (PMT), etc.

In the present exemplary embodiment, the light emission unit 241 is located above the micro-fluidic device 100 while the light receiving unit 243 corresponding to the light emission unit 241 is positioned below the micro-fluidic device 100, however, the positions of these units may be switched. Also, a light path may be adjusted using a reflecting mirror or a light guide member (not shown).

The control unit 270 controls the spindle driver 205, the data reading unit 230, the valve opening device 220 and/or the inspection unit 240 to smoothly conduct operation of the sample testing apparatus. The control unit 270 searches the diagnostic DB 260 and compares information detected in the inspection unit 240 with the diagnostic DB 260 so as to determine whether disease is found in a blood sample contained in the reaction chamber 80 of the micro-fluidic device 100.

The input unit 210 inputs measurable inspection items based on sample types fed and/or injected into the micro-fluidic device 100. The input unit 210 may be a touch screen-type device mounted on the sample testing apparatus.

For instance, if a particular sample is selected on a screen for inputting sample type, measurable inspection items using the selected sample are displayed on the screen. Then, inputting at least one selected among the displayed inspection items on the screen, the control unit 270 operates the sample testing apparatus based on the input item. In other words, where an anti-coagulant is required for inspecting a particular sample and/or testing inspection items input through the input unit 210, the valve is opened and the anti-coagulant in the anti-coagulant chamber 50 is admixed with the sample in the sample chamber 40.

The output unit 250 outputs diagnosis results and information as to whether the diagnosis is completed or not, and may include a visible output device such as a liquid crystal display (LCD), an audio output device such as a speaker, or an audiovisual output device.

The following description will be given of a control method of the micro-fluidic device according to an exemplary embodiment, using a sample testing apparatus.

After introducing a sample into the sample chamber 40 of the micro-fluidic device 100, the micro-fluidic device 100 is loaded on the sample testing apparatus. Then, by inputting a type of the sample injected into the sample chamber 40 and/or a measurable inspection item through the input unit 210, the data reading unit 230 detects the data region 11 and transfers information to the control unit 270 as to types of anti-coagulant contained in the anti-coagulant chamber 50. Then, according to the information input through the input unit 210, if the type of the anti-coagulant required for inspection is substantially identical to the type of the anti-coagulant received in the micro-fluidic device 100 which was read by the data reading unit 230, the valve 70 is opened by driving the valve opening device 220. The anti-coagulant flows into the sample chamber using the centrifugal force generated by driving the spindle motor 205.

Hereinafter, the following description will be given of a method of controlling the micro-fluidic device using a sample testing apparatus, according to another exemplary embodiment illustrated in FIG. 4.

After introducing different samples into the sample chambers 40a and 40b of the micro-fluidic device 100′, the micro-fluidic device 100′ is loaded on the sample testing apparatus. Then, by inputting the types of samples injected into the sample chambers 40a and 40b and/or measurable inspection items through the input unit 210, the data reading unit 230 detects the data region 11 and transfers information on the types of the anti-coagulants contained in the anti-coagulant chambers 50a and 50b to the control unit 270. Then, according to the information input through the input unit 210, if the type of anti-coagulant required for inspection is substantially identical to the type of anti-coagulant received in the micro-fluidic device 100′ read by the data reading unit 230, the valves 70a and 70b are opened by driving the valve opening device 220. The anti-coagulants flow into the sample chambers 40a and 40b, using the centrifugal force generated by driving the spindle motor 205. Here, the samples may be fed into either the sample chamber 40a or 40b and, in this embodiment, the sample testing apparatus may be driven by the sample operating process as described in the foregoing exemplary embodiment.

Accordingly, by introducing different samples into a single micro-fluidic device at the same time and admixing an anti-coagulant with a corresponding one of the samples, multiple samples may be tested by driving a sample testing apparatus only one time.

Hereinafter, the following description will be given method of controlling the micro-fluidic device using a sample testing apparatus, according to a further exemplary embodiment illustrated in FIG. 5.

After introducing a sample into the sample chamber 40 of the micro-fluidic device 100″, the micro-fluidic device 100″ is loaded on the sample testing apparatus. Then, by inputting a type of the sample injected into the sample chamber 40 and/or measurable inspection items through the input unit 210, the data reading unit 230 detects the data region 11 and transfers information as to types of anti-coagulants contained in the anti-coagulant chambers 51, 52 and 53 to the control unit 270. Then, according to the information input through the input unit 210, a valve of one among the anti-coagulant chambers 51, 52 and 53 is opened by driving the valve opening device 220 and the anti-coagulant flows into the sample chamber 40, using the centrifugal force generated by driving the spindle motor 205. The valve of the chamber that is opened is one for which the anti-coagulant is substantially identical to the kind of the anti-coagulant required for inspection.

Consequently, various anti-coagulants may be received in a single micro-fluidic device and, if necessary, one of the anti-coagulants corresponding to the sample may be admixed with the sample, thereby enabling a variety of inspections of a sample.

Although a few exemplary embodiments have been shown and described in conjunction with accompanying drawings, it is clearly understood that the foregoing embodiments have been proposed for illustrative purpose only and do not particularly restrict the scope of the inventive concept. Accordingly, it would be appreciated by those skilled in the art that various substitutions, variations and/or modifications may be made in these embodiments and such exemplary embodiments are not particularly restricted to particular configurations and/or arrangements described or illustrated above.

What is claimed is:

1. A micro-fluidic device comprising:
a sample chamber which receives a sample;
an anti-coagulant chamber which receives an anti-coagulant;
a channel which communicably connects the sample chamber to the anti-coagulant chamber; and
a valve which opens and closes the channel

2. The micro-fluidic device according to claim 1, wherein the micro-fluidic device is a disk-shaped platform.

3. The micro-fluidic device according to claim 2, wherein when the valve is open, the anti-coagulant in the anti-coagulant chamber flows into the sample chamber by centrifugal force generated by rotation of the micro-fluidic device.

4. The micro-fluidic device according to claim 1, wherein when the valve is open, the anti-coagulant in the anti-coagulant chamber is admixed with the sample in the sample chamber.

5. The micro-fluidic device according to claim 1, wherein the valve includes a phase transition material which is in a solid state at room temperature and is transformed into a liquid phase when heated to open the valve upon the application of heat.

6. The micro-fluidic device according to claim 5, wherein the valve further includes a micro-exothermic material which is dispersed in the phase transition material and absorbs electromagnetic radiation to emit heat energy.

7. The micro-fluidic device according to claim 1, wherein the sample chamber includes an inlet through which the sample is introduced into the sample chamber.

8. The micro-fluidic device according to claim 1 further comprising a plurality of anti-coagulant chambers, and a plurality of channels, wherein a number of channels corresponds to a number of anti-coagulant chambers, so that each sample chamber is in fluid communication with a respective anti-coagulant chamber.

9. The micro-fluidic device according to claim 8, wherein at least two of the plurality of anti-coagulant chambers receive different types of anti-coagulants.

10. The micro-fluidic device according to claim 8 further comprising providing the valve in each of the plurality of channels.

11. The micro-fluidic device according to claim 1 further comprising a plurality of sample chambers, any one of which is in fluid communication with the anti-coagulant chamber.

12. The micro-fluidic device according to claim 1 further comprising a plurality of sample chambers, a plurality of anti-coagulant chambers and a plurality of channels, wherein the plurality of channels communicably connect the plurality of sample chambers to the plurality of anti-coagulant chambers, respectively.

13. The micro-fluidic device according to claim 12 further comprising a plurality of valves provided in the plurality of channels.

14. The micro-fluidic device according to claim 13, wherein at least two of the plurality of valves are independently driven.

15. The micro-fluidic device according to claim 12, wherein at least two of the plurality of anti-coagulant chambers receive different anti-coagulants.

16. The micro-fluidic device according to claim 1 further comprising a data region which stores information regarding types of anti-coagulant contained in the anti-coagulant chamber.

17. The micro-fluidic device according to claim 16, wherein the data region includes barcode type data.

18. The micro-fluidic device according to claim 1, wherein the valve is normally closed.

19. A micro-fluidic device comprising:
a sample chamber which receives a sample;
an anti-coagulant chamber which is communicably connected to the sample chamber and receives an anti-coagulant; and
a normally closed valve which is provided between the sample chamber and the anti-coagulant chamber and is opened to selectively admix the anti-coagulant with the sample.

20. A sample testing apparatus comprising:
a micro-fluidic device that comprises a sample chamber which receives a sample, an anti-coagulant chamber which is communicably connected to the sample chamber and receives an anti-coagulant, and a normally closed valve which is provided between the sample chamber and the anti-coagulant chamber;
a valve-opening device which opens the valve; and
a control unit which drives the valve-opening device in order to selectively admix the sample in the sample chamber with the anti-coagulant from the anti-coagulant chamber.

21. The sample testing apparatus according to claim 20, wherein the valve further comprises a phase transition material which is transformed into a liquid phase when heated, and wherein the valve opening device is a heater to heat the phase transfer material.

22. The sample testing apparatus device according to claim 20, wherein the valve contains a phase transition material and a micro-exothermic material which is dispersed in the phase transition material and absorbs electromagnetic radiation to emit heat energy, and the valve opening device is an electromagnetic radiation generator.

23. The sample testing apparatus according to claim 20, wherein the micro-fluidic device is a centrifugal disk-type micro-fluidic device, and the apparatus further includes a spindle motor which rotates the micro-fluidic device.

24. The sample testing apparatus according to claim 23, wherein the control unit drives the spindle motor by centrifugal force, so that the anti-coagulant from the anti-coagulant chamber flows to the sample chamber when the valve is open.

25. The sample testing apparatus according to claim 20 further comprising an input unit which inputs information on sample types injected into the sample chamber,
wherein the control unit determines whether the anti-coagulant is required based on information on the type of sample stored in the input unit, and the control unit drives the valve opening device based on the determined results.

26. The sample testing apparatus according to claim 20 further comprising:
a plurality of anti-coagulant chambers, wherein at least two of the plurality of anti-coagulant chambers receive different anti-coagulants; and
an input unit which inputs inspection items of the micro-fluidic device, wherein the control unit drives the valve opening device to admix the sample with a corresponding one of the anti-coagulants along with the inspection items input by the input unit.

27. The sample testing apparatus according to claim 20, wherein the micro-fluidic device further comprises a data region which stores information on types of anti-coagulants contained in the anti-coagulant chamber, and a data reading unit which extracts information stored in the data region as to the types of anti-coagulant.
